(19) United States
(12) Patent Application Publication
(10) Pub. No.: US 2021/0316052 A1
(43) Pub. Date: Oct. 14, 2021

(54) Treatment of Renal Failure in House Pets using Hemodialysis

(71) Applicant: ALIPOV Aleksandr Andreevich, St. Petersburg (RU)

(72) Inventor: ALIPOV Aleksandr Andreevich, St. Petersburg (RU)

(21) Appl. No.: 17/273,027

(22) PCT Filed: Nov. 26, 2019
(86) PCT No.: PCT/RU2019/000851

§ 371 Date: Mar. 3, 2021

(30) Foreign Application Priority Data
Dec. 5, 2018 (RU) 2018142912

(51) Int. Cl. A61M 1/16; A61M 1/30; A61M 1/36

(52) U.S. Cl. CPC A61M 1/1621; A61M 1/308; A61M 2250/00; A61M 2202/0498; A61M 1/3672

(57) ABSTRACT

The invention relates to veterinary science. An extrarenal blood purification procedure is performed to remove toxins from the blood. Baseline parameters of urea and creatinine in the blood serum are measured during the blood purification procedure; on the basis of the data obtained, a dialysis intensity index (DII) is determined as the ratio of the patient's total fluid volume to the volume of the fluid purified of toxins and a uremic toxin accumulation index (UTAI) is determined as the rate of accumulation of the quantity of urea or creatinine per unit of time. Moreover, it is deemed necessary to repeat the hemodialysis procedure if the UTAI value for urea is more than 0.5 and/or the UTAI value for creatinine is more than 8, and/or the DII value is less than 0.9.

### CROSS-REFERENCE TO RELATED APPLICATIONS

This Non-Provisional Utility patent application is a U.S. National Stage Application of PCT Application Serial No. PCT/RU2019/000851, filed Nov. 26, 2019, entitled “Treatment of Renal Failure in House Pets using Hemodialysis,” which claims priority to Russian Patent Application Serial No. 2018142912, filed Dec. 5, 2018, entitled “Treatment of Renal Failure in House Pets using Hemodialysis,” the entire contents of both applications of which are hereby incorporated herein by reference.

### FIELD OF THE INVENTION

The invention relates to the veterinary field, particularly to the methods of comprehensive treatment, and can be used for acute renal failure (ARF), chronic kidney disease (CKD), and the poisoning of house pets (cats or dogs).

### BACKGROUND OF THE INVENTION

The patent RU 2636850 “The method of hemodialysis for house pets as cats and dogs” is known. It is the following: a sick cat or dog weighing up to 8 kg is connected to the extracorporeal contour, the purified blood is directed through the extracorporeal contour's blood-conducting line to the dialyzer, and after the dialyzer, the animal's purified blood is returned through the blood-conducting line. This method makes it possible to clear the blood of cats and dogs weighing up to 8 kg from uremic toxins and to stabilize the blood with an acid-ionic composition.

### SUMMARY

The main problem the invention addresses is the development of a method for treating the affected organ (the kidney). At the moment, when the physician performs nephroprotective actions with the affected organ to prevent the animal's death from uremic intoxication, the procedure of extrarenal blood purification for the animal is carried out with the aid of the “artificial kidney,” i.e., hemodialysis.

The difference with the present invention is that hemodialysis is not an aim by itself, but it is only a means of prolonging the patient's life while nephroprotection is performed.

The aim of the invention is to monitor the change in the filtration capacity of the patient's kidneys and to predict the need for repetition of the following hemodialysis procedure.

### DETAILED DESCRIPTION

The decision to assign the hemodialysis procedure is made by the physician on the basis of data from the physical examination of the animal and anamnesis. In cases where a physician concludes that the kidneys of a sick animal are not able to adequately remove the necessary amount of toxins, a decision is made to purify the organism from toxins with the aid of the “artificial kidney,” i.e., the hemodialysis procedure. The procedure is as follows: the sick animal's blood with its' toxins is pumped through the PVC tube into the apparatus; there, it is filtered from toxins, and after that, it is returned to the sick animal's bloodstream.

Treatment of renal failure in house pets using hemodialysis consists of carrying out the “extra kidney” procedure to remove toxins from the blood, measurement of the main indicators of urea and blood serum creatinine during the blood purification procedure, estimating, on the basis of experimental data, the dialysis intensity index (DII) and the rising of the uremic toxins index (RUTI) as the amount of urea or creatinine per unit of time increases.

The necessity of hemodialysis procedure repetition is defined when the RUTI of urea more than 0.5; at the RUTI of creatinine above eight, the procedure should be repeated and, regardless of the level of the RUTI, the blood purification procedure is repeated if the DII is less than 0.9.

During the procedure, to prevent thrombosis, the animal is given the anticoagulant dalteparin sodium intravenously by stream infusion 35 mouse unit (MU) per kg of body weight, and then intravenously by a drop infusion at a rate of 15 MU per kg per hour.

Depending on the condition of the patient, the physician prescribes a certain amount of liquid for the purification of toxins.

The DII is a dialysis intensity index; it is introduced to measure the effectiveness of the dialysis procedure.

If a physician determines, on the basis of a patient's condition, the amount of liquid to be purified from toxins, the DII may vary from 1 to 0.5. If the DII is less than 0.9 after that, the blood purification procedure is repeated.

The DII indicator is the ratio of the total volume of the patient's fluid, which includes blood and water, to the volume of the toxin-free fluid.

For example, a dog weighing 10 kg has six liters of fluid in its system. If all the fluid (6 liters) has been cleaned of its toxins, the index is 1. If half of the fluid (3 liters) has been cleaned, then the DII is 0.5.

The patient's fluid volume is assumed to be 65% of the body mass. The DII is represented by a decimal point. DII—0.6 means that 60% of the body's fluid was passed through the hemodialysis apparatus during the procedure.

In order to predict the re-conduct of the next hemodialysis, the RUTI has been introduced (rising of uremic toxins index), which is measured in units per hour.

Serum urea, serum creatinine, or a combination of the two can be used to calculate RUTI.

The RUTI indicator is determined by calculating the amount of increase of urea units in the blood serum per unit of time.

If the ratio is from 0.5 to 2 units per hour, the time at which the hemodialysis should be repeated shall not exceed 24 hours.

The RUTI for the creatinine is also determined by calculating the amount of increase of the units of creatinine in the blood serum per unit of time. As a rule, the amount of creatinine is 200-300 units/hour (depending on the muscular mass and the intensity of the physical load). In the case of RUTI for creatinine at levels above 8, the hemodialysis procedure should be repeated.

During acute renal failure (ARF), the kidneys do not start functioning immediately after hemodialysis. They require time to recover. Serum urea, serum creatinine, or a combination of the two can be used to calculate RUTI.

For example: after hemodialysis, the patient's urea serum was 20 mmol/1. After 24 hours, the urea serum became 32 mmol/1. In 24 hours, it had grown in 12 units. The patient's RUTI would be 0.5 unit/h. Normally, in a healthy organism, urea and creatinine RUTI is 0 unit/hour. If the kidneys are completely dysfunctional, RUTI in urea is 0.5-2 units/hour (depending on the metabolic rate and protein level of the diet).

The RUTI measurement at each inter-dialytic interval allows changes to be seen in the filtration capacity of the kidneys and predicts the need for repetition of the next hemodialysis.

### Variants for Carrying Out the Invention

### Example 1

Species: dog, Name: Lex, Breed: Metis, Gender: Male, Weight: 20 kg, Diagnosis: pyroplasmosis with ARF.

### Day 1

| | |
|---|---|
| Animal weight, kg | 20 |
| Volume of exchange fluid of the body, liters | 13 |
| DII indicator | 0.65 |
| RUTI indicator of urea, unit/hour | — |
| RUTI indicator of creatinine, unit/hour | — |
| Creatinine before hemodialysis, mcmo1/1 | 1803 |
| Creatinine after hemodialysis, mcmo1/1 | 632 |
| Urea before hemodialysis, mmol/1 | 76 |
| Urea after hemodialysis, mmol/1 | 27 |

The RUTI indicators for urea and creatinine are impossible to calculate because the animal has undergone a primary hemodialysis procedure.

We will repeat the hemodialysis procedure in 24 hours since the DII rate is less than 0.9.

### Day 2

| | |
|---|---|
| Animal weight, kg | 20 |
| Volume of exchange fluid of the body, liters | 13 |
| Dalteparin sodium bolus, before hemodialysis, MU | 700 |
| Dalteparin sodium, during hemodialysis, MU/hour | 300 |
| DII indicator | 0.7 |
| RUTI indicator of urea, unit/hour | 1.3 |
| RUTI indicator of creatinine, unit/hour | 23 |
| Creatinine before hemodialysis, mcmo1/1 | 1184 |
| Creatinine after hemodialysis, mcmo1/1 | 687 |
| Urea before hemodialysis, mmol/1 | 58 |
| Urea after hemodialysis, mmol/1 | 18 |

We will repeat the purification procedure in 24 hours, as it is necessary because of RUTI and DII indicators.

### Day 3

| | |
|---|---|
| Animal weight, kg | 20 |
| Volume of exchange fluid of the body, liters | 13 |
| Dalteparin sodium bolus, before hemodialysis, MU | 700 |
| Dalteparin sodium, during hemodialysis, MU/hour | 300 |
| DII indicator | 0.7 |
| RUTI indicator of urea, unit/hour | 1.1 |
| RUTI indicator of creatinine, unit/hour | 19 |
| Creatinine before hemodialysis, mcmo1/1 | 1143 |
| Creatinine after hemodialysis, mcmo1/1 | 286 |
| Urea before hemodialysis, mmol/1 | 44 |
| Urea after hemodialysis, mmol/1 | 18 |

We will repeat the purification procedure in 24 hours, as it is necessary because of RUTI and DII indicators.

### Day 4

| | |
|---|---|
| Animal weight, kg | 20 |
| Volume of exchange fluid of the body, liters | 13 |
| Dalteparin sodium bolus, before hemodialysis, MU | 700 |
| Dalteparin sodium, during hemodialysis, MU/hour | 300 |
| DII indicator | 0.7 |
| RUTI indicator of urea, unit/hour | 0.5 |
| RUTI indicator of creatinine, unit/hour | 12 |
| Creatinine before hemodialysis, mcmo1/1 | 574 |
| Creatinine after hemodialysis, mcmo1/1 | 230 |
| Urea before hemodialysis, mmol/1 | 30 |
| Urea after hemodialysis, mmol/1 | 12 |

We will repeat the purification procedure in 24 hours, as it is necessary because of RUTI and DII indicators.

### Day 5

Due to the negative RUTI indicator for creatinine (−4) and a very small RUTI indicator for urea (0.12), it was concluded that hemodialysis was not necessary for the animal. The animal was discharged in 24 hours.

| | |
|---|---|
| Animal weight, kg | 20 |
| Volume of exchange fluid of the body, liters | 13 |
| DII indicator | 1 |
| RUTI indicator of urea, unit/hour | 0.12 |
| RUTI indicator of creatinine, unit/hour | -4 |
| Creatinine 24 hours after hemodialysis, mcmo1/1 | 134 |
| Urea 24 hours after hemodialysis, mcmo1/1 | 15 |

### Example 2

Species: dog, Name: Puma, Breed: AmStaff, Gender: Female, Weight: 28 kg, Diagnosis: Acute pyelonephritis

### Day 1

| | |
|---|---|
| Animal weight, kg | 28 |
| Volume of exchange fluid of the body, liters | 18 |
| Dalteparin sodium bolus, before hemodialysis, MU | 980 |
| Dalteparin sodium, during hemodialysis, MU/hour | 420 |
| DII indicator | 0.5 |
| RUTI indicator of urea, unit/hour | — |
| RUTI indicator of creatinine, unit/hour | — |
| Creatinine before hemodialysis, mcmo1/1 | 1234 |
| Creatinine after hemodialysis, mcmol/1 | 617 |
| Urea before hemodialysis, mmo1/1 | 53 |
| Urea after hemodialysis, mmo1/1 | 27 |

RUTI indicators for urea and creatinine cannot be calculated because the animal has undergone a primary hemodialysis procedure.

We will repeat hemodialysis in 24 hours, as DII indicator is less than 0.9.

### Day 2

Due to the negative RUTI indicator for creatinine (−13) and a negative RUTI indicator for urea (−0.3), it was concluded that hemodialysis was not necessary for the animal. The animal was discharged.

| | |
|---|---|
| Animal weight, kg | 28 |
| Volume of exchange fluid of the body, liters | 18 |
| DII indicator | — |
| RUTI indicator of urea, unit/hour | -0.3 |
| RUTI indicator of creatinine, unit/hour | -13 |
| Creatinine 24 hours after hemodialysis, mcmo1/1 | 305 |
| Urea 24 hours after hemodialysis, mcmo1/1 | 20 |

### Example 3

Species: dog, Name: Bait, Breed: Badger-dog, Gender: Male, Weight: 9 kg, Diagnosis: exacerbation of chronic interstitial nephritis and acute dehydration, affected by debilitating vomiting.

### Day 1

| | |
|---|---|
| Animal weight, kg | 9 |
| Volume of exchange fluid of the body, liters | 6 |
| Dalteparin sodium bolus, before hemodialysis, MU | 315 |
| Dalteparin sodium, during hemodialysis, MU/hour | 135 |
| DII indicator | 0.4 |
| RUTI indicator of urea, unit/hour | — |
| RUTI indicator of creatinine, unit/hour | — |
| Creatinine before hemodialysis, mcmo1/1 | 980 |
| Creatinine after hemodialysis, mcmo1/1 | 392 |
| Urea before hemodialysis, mmol/1 | 49 |
| Urea after hemodialysis, mmol/1 | 29 |

RUTI indicators for urea and creatinine cannot be calculated because the animal has undergone a primary hemodialysis procedure.

We will repeat hemodialysis in 24 hours, as DII indicator is less than 0.9.

### Day 2

| | |
|---|---|
| Animal weight, kg | 9 |
| Volume of exchange fluid of the body, liters | 6 |
| Dalteparin sodium bolus, before hemodialysis, MU | 315 |
| Dalteparin sodium, during hemodialysis, MU/hour | 135 |
| DII indicator | 0.6 |
| RUTI indicator of urea, unit/hour | 0.7 |
| RUTI indicator of creatinine, unit/hour | 11 |
| Creatinine before hemodialysis, mcmo1/1 | 656 |
| Creatinine after hemodialysis, mcmo1/1 | 394 |
| Urea before hemodialysis, mmol/1 | 46 |
| Urea after hemodialysis, mmol/1 | 28 |

We will repeat the purification procedure in 24 hours, as it is necessary because of RUTI and DII indicators.

### Day 3

| | |
|---|---|
| Animal weight, kg | 9 |
| Volume of exchange fluid of the body, liters | 6 |
| Dalteparin sodium bolus, before hemodialysis, MU | 315 |
| Dalteparin sodium, during hemodialysis, MU/hour | 135 |
| DII indicator | 0.6 |
| RUTI indicator of urea, unit/hour | 0.2 |
| RUTI indicator of creatinine, unit/hour | 4 |
| Creatinine before hemodialysis, mcmo1/1 | 490 |
| Creatinine after hemodialysis, mcmo1/1 | 196 |
| Urea before hemodialysis, mmol/1 | 33 |
| Urea after hemodialysis, mmol/1 | 13 |

We will repeat the purification procedure in 24 hours.

### Day 4

Due to the negative RUTI indicator for creatinine (−2) and a small RUTI indicator for urea (0.2), it was concluded that hemodialysis was not necessary for the animal. The animal was discharged two days after the procedure.

| | |
|---|---|
| Animal weight, kg | 9 |
| Volume of exchange fluid of the body, liters | 6 |
| DII indicator | — |
| RUTI indicator of urea, unit/hour | 0.2 |
| RUTI indicator of creatinine, unit/hour | 1 |
| Creatinine 24 hours after hemodialysis, mcmo1/1 | 220 |
| Urea 24 hours after hemodialysis, mcmo1/1 | 18 |

As can be seen from the given examples, the RUTI measurement for urea and creatinine at each inter-dialytic interval allows for seeing changes in the filtration capacity of the kidneys and predicting the need for repetition of hemodialysis or the absence of such a need.

The use of this procedure significantly increases the animal's lifetime. During the procedure, the physician can medically correct renal pathology when the kidneys have temporarily lost their function—to clear the organism from uremic toxins.

1. A method of treating renal failure in house pets using hemodialysis, comprising:
Implementing a procedure for external purification of blood from toxins;
measuring key indicators of urea and creatine of blood serum within a blood purification process;
based on data obtained from the measuring of the key indicators—defining a dialysis intensity index (DII) and rising of uremic toxins index (RUTI), which is defined as rising amounts of urea or creatine per unit of time; and
defining a necessity of a repeated procedure of hemodialysis when a RUTI indicator for urea is more than 0.5.

2. The method of claim 1, wherein, during the procedure, in order to prevent thrombosis, an animal is given an anticoagulant Dalteparin sodium, intravenously by stream infusion 35 mouse unit (MU) per kg of body weight, and then intravenously by a drop infusion at a rate of 15 MU per kg per hour.

3. The method of claim 1, wherein defining a necessity of a repeated procedure of hemodialysis when the RUTI indicator for creatine is more than 8.

4. The method of claim 1, wherein defining a necessity of a repeated procedure of hemodialysis when the DII indicator is less than 0.9.
